(19) United States
(12) Patent Application Publication
(10) Pub. No.: US 2008/0303425 A1
(43) Pub. Date: Dec. 11, 2008

(54) CHRYSENES FOR BLUE LUMINESCENT APPLICATIONS

(76) Inventors: VSEVOLOD ROSTOVTSEV, Swarthmore, PA (US); Hong Meng, Wilmington, DE (US); Lois Bryman, West Chester, PA (US); Jeffrey A. Merlo, Wilmington, DE (US); Norman Herron, Newark, DE (US); Reid John Chesterfield, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/121,883

(22) Filed: May 16, 2008

Related U.S. Application Data
(60) Provisional application No. 60/941,404, filed on Jun. 1, 2007.

(51) Int. Cl. H01L 51/52; C07C 211/61

(52) U.S. Cl. 313/504; 564/426

(57) ABSTRACT

This invention relates to chrysene compounds that are useful in electroluminescent applications and are capable of blue emission. It also relates to electronic devices in which the active layer includes such a chrysene compound.

### RELATED APPLICATION DATA

This application claims priority under 35 U.S.C. § 119(e) from U.S. Provisional Application No. 60/941,404 filed on Jun. 1, 2007, which is incorporated by reference herein in its entirety.

### BACKGROUND

1. Field of the Disclosure

This invention relates to electroluminescent chrysene compounds which have blue emission. It also relates to electronic devices in which the active layer includes such a chrysene compound.

2. Description of the Related Art

Organic electronic devices that emit light, such as light-emitting diodes that make up displays, are present in many different kinds of electronic equipment. In all such devices, an organic active layer is sandwiched between two electrical contact layers. At least one of the electrical contact layers is light-transmitting so that light can pass through the electrical contact layer. The organic active layer emits light through the light-transmitting electrical contact layer upon application of electricity across the electrical contact layers.

It is well known to use organic electroluminescent compounds as the active component in light-emitting diodes. Simple organic molecules such as anthracene, thiadiazole derivatives, and coumarin derivatives are known to show electroluminescence. Semiconductive conjugated polymers have also been used as electroluminescent components, as has been disclosed in, for example, U.S. Pat. No. 5,247,190, U.S. Pat. No. 5,408,109, and Published European Patent Application 443 861.

However, there is a continuing need for electroluminescent compounds, especially compounds that are blue-emitting.

### SUMMARY

There is provided a compound having Formula I:

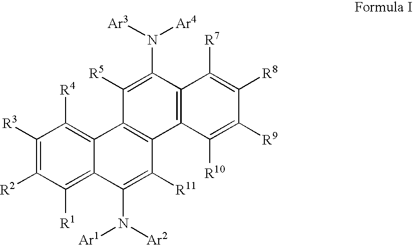

wherein:
   Ar1 through Ar4 are the same or different and are aryl, and at least one of Ar1 through Ar4 is substituted;
   R1 through R5 and R7 through R11 are the same or different and are selected from the group consisting of H and a branched alkyl, or adjacent R groups may be joined together to form a 5- or 6-membered aliphatic ring, with the proviso that either (i) R3 is a branched alkyl or (ii) R2 and R3 together form a 5- or 6-membered aliphatic ring;
wherein said compound is capable of emitting blue light.

There is also provided an electronic device comprising an active layer comprising the compound of Formula I.

### BRIEF DESCRIPTION OF THE DRAWINGS

Embodiments are illustrated in the accompanying figures to improve understanding of concepts as presented herein.

FIG. 1 includes an illustration of one example of an organic electronic device.

Skilled artisans appreciate that objects in the figures are illustrated for simplicity and clarity and have not necessarily been drawn to scale. For example, the dimensions of some of the objects in the figures may be exaggerated relative to other objects to help to improve understanding of embodiments.

### DETAILED DESCRIPTION

Many aspects and embodiments are disclosed herein and are exemplary and not limiting. After reading this specification, skilled artisans appreciate that other aspects and embodiments are possible without departing from the scope of the invention.

Other features and benefits of any one or more of the embodiments will be apparent from the following detailed description, and from the claims. The detailed description first addresses Definitions and Clarification of Terms followed by the Chrysene Compound, the Electronic Device, and finally Examples.

### 1. Definitions and Clarification of Terms

Before addressing details of embodiments described below, some terms are defined or clarified.

As used herein, the term “compound” is intended to mean an electrically uncharged substance made up of molecules that further consist of atoms, wherein the atoms cannot be separated by physical means. The phrase “adjacent to,” when used to refer to layers in a device, does not necessarily mean that one layer is immediately next to another layer. On the other hand, the phrase “adjacent R groups,” is used to refer to R groups that are next to each other in a chemical formula (i.e., R groups that are on atoms joined by a bond). The term “photoactive” refers to any material that exhibits electroluminescence and/or photosensitivity.

The term “aryl” is intended to mean a group derived from an aromatic hydrocarbon having one point of attachment. The term includes groups which have a single ring and those which have multiple rings which can be joined by a single bond or fused together. The term is intended to include heteroaryls. The term “arylene” is intended to mean a group derived from an aromatic hydrocarbon having two points of attachment. In some embodiments, an aryl group has from 3-60 carbon atoms.

The term “alkyl” is intended to mean a group derived from an aliphatic hydrocarbon having one point of attachment, and includes a linear, a branched, or a cyclic group. The term is intended to include heteroalkyls. The term “alkylene” is intended to mean a group derived from an aliphatic hydrocarbon and having two or more points of attachment. In some embodiments, an alkyl group has from 1-20 carbon atoms.

The term “branched alkyl” refers to an alkyl group having at least one secondary or tertiary carbon. The term “secondary alkyl” refers to a branched alkyl group having a secondary carbon atom. The term “tertiary alkyl” refers to a branched alkyl group having a tertiary carbon atom. In some embodiments, the branched alkyl group is attached via a secondary or tertiary carbon.

The term “aliphatic ring” is intended to mean a cyclic group that does not have delocalized pi electrons. In some embodiments, the aliphatic ring has no unsaturation. In some embodiments, the ring has one double or triple bond.

The term “binaphthyl” is intended to mean a group having two naphthalene units joined by a single bond. In some embodiments, the binaphthyl group is 1,1-binaphthyl, which is attached at the 3-, 4-, or 5-position; in some embodiments, 1,2-binaphthyl, which is attached at the 3-, 4-, or 5-position on the 1-naphthyl moiety, or the 4- or 5-position on the 2-naphthyl moiety; and in some embodiments, 2,2-binaphthyl, which is attached at the 4- or 5-position.

The term “biphenyl” is intended to mean a group havin two phenyl units joined by a single bond. The group can be attached at the 2-, 3-, or 4-position.

The term “blue” refers to radiation that has an emission maximum at a wavelength in a range of approximately 400-500 nm.

The prefix “hetero” indicates that one or more carbon atoms have been replaced with a different atom. In some embodiments, the different atom is N, O, or S.

All groups may be unsubstituted or substituted. In some embodiments, the substituents are selected from the group consisting of halide, alkyl, alkoxy, aryl, and cyano.

The term “layer” is used interchangeably with the term “film” and refers to a coating covering a desired area. The term is not limited by size. The area can be as large as an entire device or as small as a specific functional area such as the actual visual display, or as small as a single sub-pixel. Layers and films can be formed by any conventional deposition technique, including vapor deposition, liquid deposition (continuous and discontinuous techniques), and thermal transfer. Continuous deposition techniques, include but are not limited to, spin coating, gravure coating, curtain coating, dip coating, slot-die coating, spray coating, and continuous nozzle coating. Discontinuous deposition techniques include, but are not limited to, ink jet printing, gravure printing, and screen printing.

The term “organic electronic device” or sometimes just “electronic device” is intended to mean a device including one or more organic semiconductor layers or materials.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The IUPAC numbering system is used throughout, where the groups from the Periodic Table are numbered from left to right as 1-18 (CRC Handbook of Chemistry and Physics, 81^{st} Edition, 2000).

### 2. Chrysene Compound

One aspect of the present invention is a composition of Formula I:

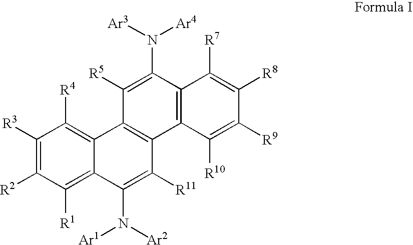

wherein:
   Ar1 through Ar4 are the same or different and are aryl, and at least one of Ar1 through Ar4 is substituted;

R1 through R5 and R7 through R11 are the same or different and are selected from the group consisting of H and a branched alkyl, or adjacent R groups may be joined together to form a 5- or 6-membered aliphatic ring, with the proviso that either (i) R3 is a branched alkyl or (ii) R2 and R3 together form a 5- or 6-membered aliphatic ring.
The compound is capable of blue emission.

In some embodiments, the branched alkyl group has from 3-8 carbon atoms. In some embodiments, the branched alkyl group is a secondary alkyl selected from the group consisting of isopropyl and 2-butyl. In some embodiments, the branched alkyl group is a tertiary alkyl selected from the group consisting of t-butyl and 2-(2-methyl)-butyl.

In some embodiments, R3 is a branched alkyl group. In some embodiments, R1, R2, R5, and R7 through R11 are H.

In some embodiments, R2 and R3 taken together form a 5- or 6-membered aliphatic ring. In some embodiments, the aliphatic ring is selected from the group consisting of cyclohexyl and cyclopentyl. In some embodiments, the aliphatic ring has one or more alkyl substituents.

In some embodiments, Ar1 through Ar4 are independently selected from the group consisting of phenyl, biphenyl, naphthyl, binaphthyl, phenylnaphthyl, and naphthylphenyl. In some embodiments, at least one aryl group has a substituent selected from the group consisting of C1-20 alkyl, C1-20 alkoxy, perfluoroalkyl, cyano, and fluoro. In some embodiments, the alkyl, alkoxy, and perfluoroalkyl groups have 1-8 carbons.

In some embodiments, Ar1 and Ar3 are phenyl groups. In some embodiments, Ar1 and Ar3 are phenyl groups having one substituent selected from perfluoroalkyl, cyano, and fluoro. In some embodiments, the perfluoroalkyl group is trifluoromethyl. In some embodiments Ar1 and Ar3 are phenyl groups having 1-5 substituents selected from the group consisting of alkyl groups and alkoxy groups.

In some embodiments, Ar2 and Ar4 are selected from the group consisting of phenyl and biphenyl groups. In some embodiments, Ar2 and Ar4 have at least one alkyl substituent.

In some embodiments, the blue chrysene compound is selected from compounds E1 through E9:

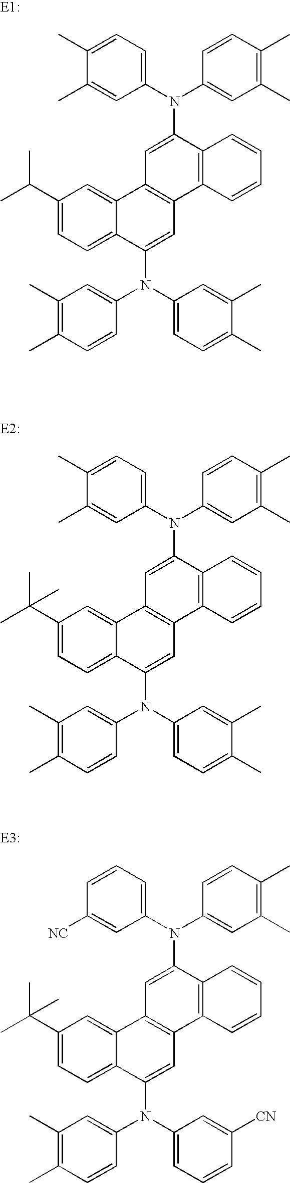

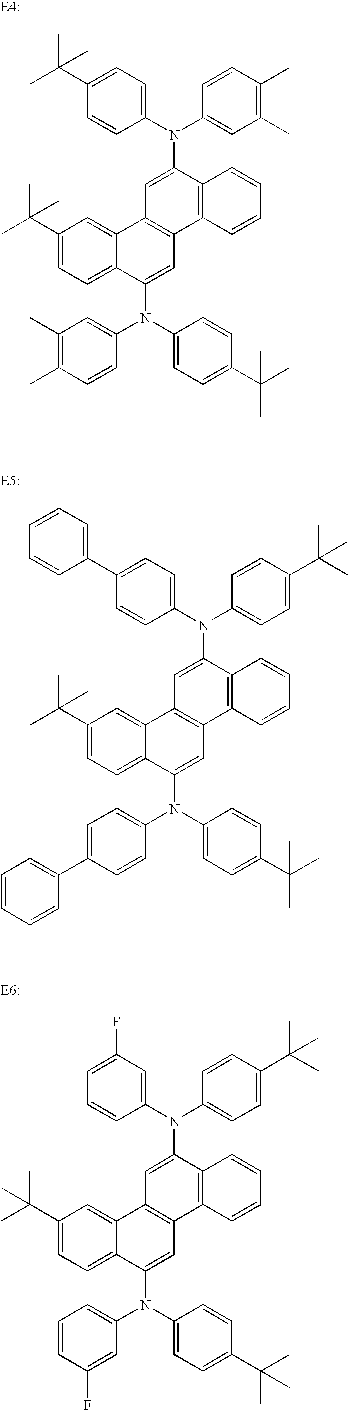

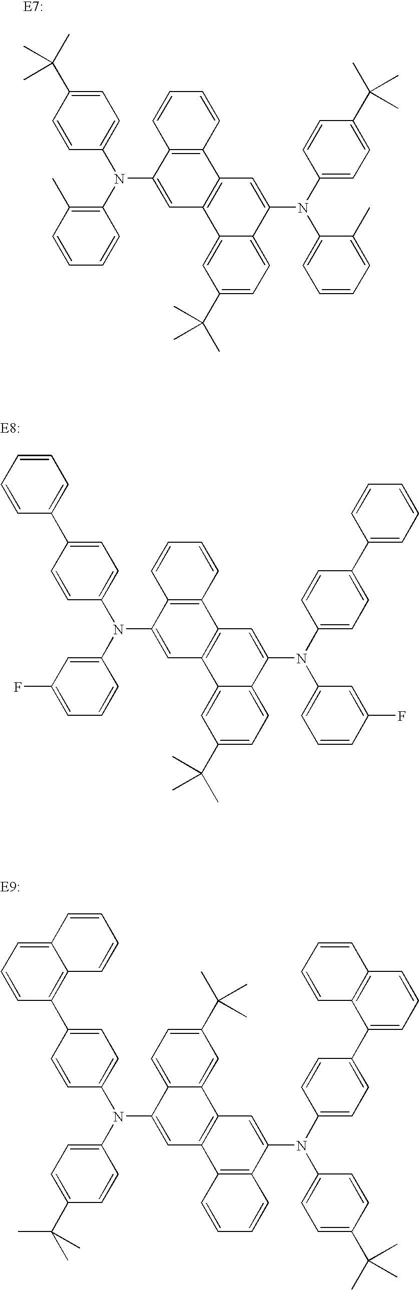

In some embodiments, the blue chrysene compound is selected from E10 through E15 below.

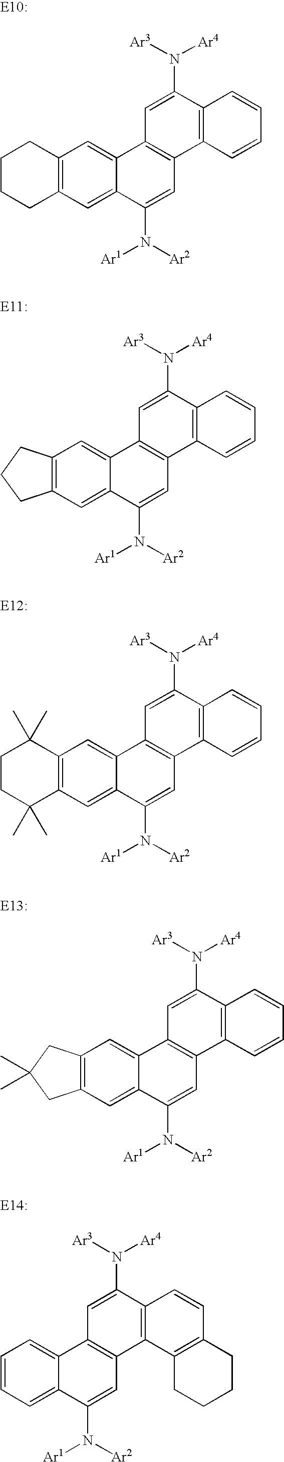

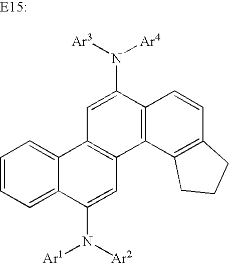

In Compounds E10 through E15, Ar1 through Ar4 are as described above. In some embodiments, Ar1 through Ar4 are selected from the group consisting of phenyl and biphenyl. In some embodiments, Ar1=Ar2 and Ar3=Ar4. In some embodiments, Ar1=Ar3 and Ar2=Ar4.

The new chrysenes can be prepared by known coupling and substitution reactions. Exemplary preparations are given in the Examples.

The chrysene compounds described herein can be formed into films using liquid deposition techniques. Thin films of these materials dispersed in a host matrix exhibit good to excellent photoluminescent properties and blue emission.

### 3. Electronic Device

Organic electronic devices that may benefit from having one or more layers comprising the blue luminescent materials described herein include, but are not limited to, (1) devices that convert electrical energy into radiation (e.g., a light-emitting diode, light emitting diode display, or diode laser), (2) devices that detect signals through electronics processes (e.g., photodetectors, photoconductive cells, photoresistors, photoswitches, phototransistors, phototubes, IR detectors), (3) devices that convert radiation into electrical energy, (e.g., a photovoltaic device or solar cell), and (4) devices that include one or more electronic components that include one or more organic semi-conductor layers (e.g., a transistor or diode).

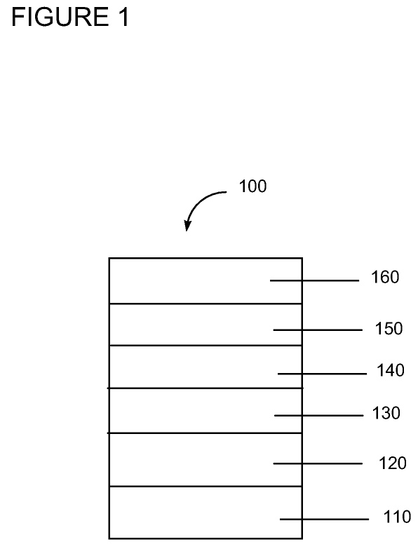

One illustration of an organic electronic device structure is shown in FIG. 1. The device 100 has a first electrical contact layer, an anode layer 110 and a second electrical contact layer, a cathode layer 160, and a photoactive layer 140 between them. Adjacent to the anode is a buffer layer 120. Adjacent to the buffer layer is a hole transport layer 130, comprising hole transport material. Adjacent to the cathode may be an electron transport layer 150, comprising an electron transport material. As an option, devices may use one or more additional hole injection or hole transport layers (not shown) next to the anode 110 and/or one or more additional electron injection or electron transport layers (not shown) next to the cathode 160.

Layers 120 through 150 are individually and collectively referred to as the active layers.

In one embodiment, the different layers have the following range of thicknesses: anode 110, 500-5000 Å, in one embodiment 1000-2000 Å; buffer layer 120, 50-2000 Å, in one embodiment 200-1000 Å; hole transport layer 130, 50-2000 Å, in one embodiment 200-1000 Å; photoactive layer 140, 10-2000 Å, in one embodiment 100-1000 Å; layer 150, 50-2000 Å, in one embodiment 100-1000 Å; cathode 160, 200-10000 Å, in one embodiment 300-5000 A. The location of the electron-hole recombination zone in the device, and thus the emission spectrum of the device, can be affected by the relative thickness of each layer. The desired ratio of layer thicknesses will depend on the exact nature of the materials used.

Depending upon the application of the device 100, the photoactive layer 140 can be a light-emitting layer that is activated by an applied voltage (such as in a light-emitting diode or light-emitting electrochemical cell), or a layer of material that responds to radiant energy and generates a signal with or without an applied bias voltage (such as in a photodetector). Examples of photodetectors include photoconductive cells, photoresistors, photoswitches, phototransistors, and phototubes, and photovoltaic cells, as these terms are described in Markus, John, Electronics and Nucleonics Dictionary, 470 and 476 (McGraw-Hill, Inc. 1966).

a. Photoactive Layer

The chrysene compounds of Formula I are useful as photoactive materials in layer 140. The compounds can be used alone, or in combination with a host material.

In some embodiments, the host is a bis-condensed cyclic aromatic compound.

In some embodiments, the host is an anthracene derivative compound. In some embodiments the compound has the formula:

An-L-An

where:

An is an anthracene moiety;

L is a divalent connecting group.

In some embodiments of this formula, L is a single bond, —O—, —S—, —N(R)—, or an aromatic group. In some embodiments, An is a mono- or diphenylanthryl moiety.

In some embodiments, the host has the formula:

A-An-A

where:

An is an anthracene moiety;

A is the same or different at each occurrence and is an aromatic group.

In some embodiments, the A groups are attached at the 9- and 10-positions of the anthracene moiety. In some embodiments, A is selected from the group consisting naphthyl, naphthylphenylene, and naphthylnaphthylene. In some embodiments the compound is symmetrical and in some embodiments the compound is non-symmetrical.

In some embodiments, the host has the formula:

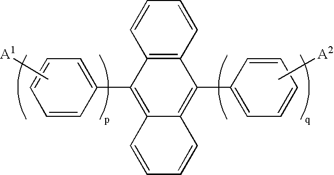

where:

A¹ and A² are the same or different at each occurrence and are selected from the group consisting of H, an aromatic group, and an alkenyl group, or A may represent one or more fused aromatic rings;

p and q are the same or different and are an integer from 1-3.

In some embodiments, the anthracene derivative is non-symmetrical. In some embodiments, p=2 and q=1. In some embodiments, at least one of A¹ and A² is a naphthyl group.

In some embodiments, the host is selected from the group consisting of

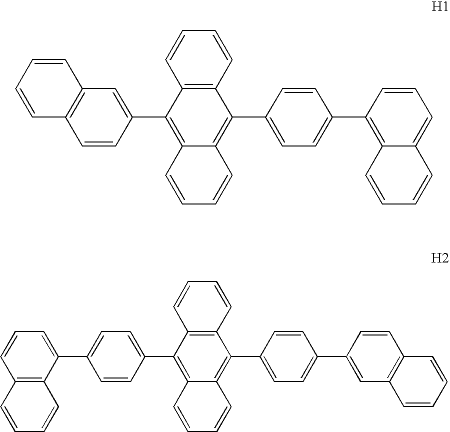

and combinations thereof.

The chrysene compounds of Formula I, in addition to being useful as emissive dopants in the photoactive layer, can also act as charge carrying hosts for other emissive dopants in the photoactive layer 140.

b. Other Device Layers

The other layers in the device can be made of any materials that are known to be useful in such layers.

The anode 110, is an electrode that is particularly efficient for injecting positive charge carriers. It can be made of, for example, materials containing a metal, mixed metal, alloy, metal oxide or mixed-metal oxide, or it can be a conducting polymer, or mixtures thereof. Suitable metals include the Group 11 metals, the metals in Groups 4-6, and the Group 8-10 transition metals. If the anode is to be light-transmitting, mixed-metal oxides of Groups 12, 13 and 14 metals, such as indium-tin-oxide, are generally used. The anode 110 can also comprise an organic material such as polyaniline as described in “Flexible light-emitting diodes made from soluble conducting polymer,” Nature vol. 357, pp 477-479 (11 Jun. 1992). At least one of the anode and cathode is desirably at least partially transparent to allow the generated light to be observed.

The buffer layer 120 comprises buffer material and may have one or more functions in an organic electronic device, including but not limited to, planarization of the underlying layer, charge transport and/or charge injection properties, scavenging of impurities such as oxygen or metal ions, and other aspects to facilitate or to improve the performance of the organic electronic device. Buffer materials may be polymers, oligomers, or small molecules. They may be vapour deposited or deposited from liquids which may be in the form of solutions, dispersions, suspensions, emulsions, colloidal mixtures, or other compositions.

The buffer layer can be formed with polymeric materials, such as polyaniline (PANI) or polyethylenedioxythiophene (PEDOT), which are often doped with protonic acids. The protonic acids can be, for example, poly(styrenesulfonic acid), poly(2-acrylamido-2-methyl-1-propanesulfonic acid), and the like.

The buffer layer can comprise charge transfer compounds, and the like, such as copper phthalocyanine and the tetrathiafulvalene-tetracyanoquinodimethane system (TTF-TCNQ).

In some embodiments, the buffer layer comprises at least one electrically conductive polymer and at least one fluorinated acid polymer. Such materials have been described in, for example, published U.S. patent applications 2004-0102577, 2004-0127637, and 2005/205860

Examples of hole transport materials for layer 130 have been summarized for example, in Kirk-Othmer Encyclopedia of Chemical Technology, Fourth Edition, Vol. 18, p. 837-860, 1996, by Y. Wang. Both hole transporting molecules and polymers can be used. Commonly used hole transporting molecules are: N,N′-diphenyl-N,N′-bis(3-methylphenyl)-[1,1′-biphenyl]-4,4′-diamine (TPD), 1,1-bis[(di-4-tolylamino) phenyl]cyclohexane (TAPC), N,N′-bis(4-methylphenyl)-N,N′-bis(4-ethylphenyl)-[1,1′-(3,3′-dimethyl)-biphenyl]-4,4′-diamine (ETPD), tetrakis-(3-methylphenyl)-N,N,N′,N′-2,5-phenylenediamine (PDA), a-phenyl-4-N,N-diphenylaminostyrene (TPS), p-(diethylamino)benzaldehyde diphenylhydrazone (DEH), triphenylamine (TPA), bis[4-(N,N-diethylamino)-2-methylphenyl](4-methylphenyl)methane (MPMP), 1-phenyl-3-[p-(diethylamino)styryl]-5-[p-(diethylamino)phenyl] pyrazoline (PPR or DEASP), 1,2-trans-bis(9H-carbazol-9-yl)cyclobutane (DCZB), N,N,N′,N′-tetrakis(4-methylphenyl)-(1,1′-biphenyl)-4,4′-diamine (TTB), N,N′-bis(naphthalen-1-yl)-N,N′-bis-(phenyl)benzidine (α-NPB), and porphyrinic compounds, such as copper phthalocyanine. Commonly used hole transporting polymers are polyvinylcarbazole, (phenylmethyl)-polysilane, and polyaniline. It is also possible to obtain hole transporting polymers by doping hole transporting molecules such as those mentioned above into polymers such as polystyrene and polycarbonate. In some cases, triarylamin polymers are used, especially triarylamine-fluorene copolymers. In some cases, the polymers and copolymers are crosslinkable.

Examples of additional electron transport materials which can be used in layer 150 include metal chelated oxinoid compounds, such as tris(8-hydroxyquinolato)aluminum (Alq₃); bis(2-methyl-8-quinolinolato)(para-phenyl-phenolato)aluminum(III) (BAIQ); and azole compounds such as 2-(4-biphenylyl)-5-(4-t-butylphenyl)-1,3,4-oxadiazole (PBD) and 3-(4-biphenylyl)-4-phenyl-5-(4-t-butylphenyl)-1,2,4-triazole (TAZ), and 1,3,5-tri(phenyl-2-benzimidazole)benzene (TPBI); quinoxaline derivatives such as 2,3-bis(4-fluorophenyl)quinoxaline; phenanthroline derivatives such as 9,10-diphenylphenanthroline (DPA) and 2,9-dimethyl-4,7-diphenyl-1,10-phenanthroline (DDPA); and mixtures thereof. Layer 150 can function both to facilitate electron transport, and also serve as a buffer layer or confinement layer to prevent quenching of the exciton at layer interfaces. Preferably, this layer promotes electron mobility and reduces exciton quenching.

The cathode 160, is an electrode that is particularly efficient for injecting electrons or negative charge carriers. The cathode can be any metal or nonmetal having a lower work function than the anode. Materials for the cathode can be selected from alkali metals of Group 1 (e.g., Li, Cs), the Group 2 (alkaline earth) metals, the Group 12 metals, including the rare earth elements and lanthanides, and the actinides. Materials such as aluminum, indium, calcium, barium, samarium and magnesium, as well as combinations, can be used. Li-containing organometallic compounds, LiF, and Li₂O can also be deposited between the organic layer and the cathode layer to lower the operating voltage.

It is known to have other layers in organic electronic devices. For example, there can be a layer (not shown) between the anode 110 and buffer layer 120 to control the amount of positive charge injected and/or to provide band-gap matching of the layers, or to function as a protective layer. Layers that are known in the art can be used, such as copper phthalocyanine, silicon oxy-nitride, fluorocarbons, silanes, or an ultra-thin layer of a metal, such as Pt. Alternatively, some or all of anode layer 110, active layers 120, 130, 140, and 150, or cathode layer 160, can be surface-treated to increase charge carrier transport efficiency. The choice of materials for each of the component layers is preferably determined by balancing the positive and negative charges in the emitter layer to provide a device with high electroluminescence efficiency.

It is understood that each functional layer can be made up of more than one layer.

The device can be prepared by a variety of techniques, including sequential vapor deposition of the individual layers on a suitable substrate. Substrates such as glass, plastics, and metals can be used. Conventional vapor deposition techniques can be used, such as thermal evaporation, chemical vapor deposition, and the like. Alternatively, the organic layers can be applied from solutions or dispersions in suitable solvents, using conventional coating or printing techniques, including but not limited to spin-coating, dip-coating, roll-to-roll techniques, ink-jet printing, screen-printing, gravure printing and the like.

The present invention also relates to an electronic device comprising at least one active layer positioned between two electrical contact layers, wherein the at least one active layer of the device includes the chrysene compound of Formula 1. Devices frequently have additional hole transport and electron transport layers.

To achieve a high efficiency LED, the HOMO (highest occupied molecular orbital) of the hole transport material desirably aligns with the work function of the anode, and the LUMO (lowest un-occupied molecular orbital) of the electron transport material desirably aligns with the work function of the cathode. Chemical compatibility and sublimation temperature of the materials are also important considerations in selecting the electron and hole transport materials.

It is understood that the efficiency of devices made with the chrysene compounds described herein, can be further improved by optimizing the other layers in the device. For example, more efficient cathodes such as Ca, Ba or LiF can be used. Shaped substrates and novel hole transport materials that result in a reduction in operating voltage or increase quantum efficiency are also applicable. Additional layers can also be added to tailor the energy levels of the various layers and facilitate electroluminescence.

The chrysene compounds of the invention often are fluorescent and photoluminescent and can be useful in applications other than OLEDs, such as oxygen sensitive indicators and as fluorescent indicators in bioassays.

### EXAMPLES

The following examples illustrate certain features and advantages of the present invention. They are intended to be illustrative of the invention, but not limiting. All percentages are by weight, unless otherwise indicated.

### Example 1

This example illustrates the preparation of Compound E1, 3-iso-propyl-N⁶,N⁶,N¹²,N¹²-tetrakis(3,4-dimethylphenyl)-chrysene-6,12-diamine

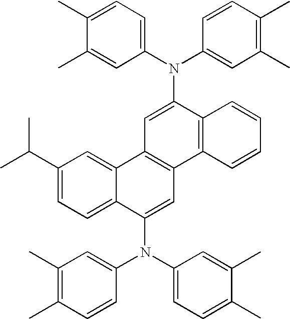

a. Preparation of (Z)-1-(4-iso-propylstyryl)naphthalene.

In a drybox, 1-vinylnaphthalene (9.80 g, 63.5 mmol) and 4-bromocumene (11.5 g, 57.8 mmol) were placed into a 250 ml RB flask and dissolved in 80 ml of dry DMF. Palladium catalyst (trans-di(μ-acetato)bis[o-(di-o-tolylphosphino)benzyl] dipalladium (II), 0.542 g, 0.58 mmol) and sodium acetate (7.11 g, 86.6 mmol) were added last, followed by 20 ml of dry DMF. The flask was capped, taken out of the box and attached to a condenser flushed with nitrogen. The reaction mixture was stirred at 120° C. for 60 hours. Solution was cooled to room temperature and poured into 750 ml of water. Aqueous layer was extracted with CH₂Cl₂ (3×500 ml). Combined organic layers were rinsed with water and brine (500 ml each), dried over MgSO₄ and volatiles were removed under vacuum. Crude product was adsorbed onto 25 g of silica, loaded onto a 4″ column and eluted with hexane first, then with 20% CH₂Cl₂ in hexane. The first fraction was concentrated and residue was dried under high vacuum to 6.3 g (40%) of the desired product. ¹H NMR is analogous to the one reported in the literature (Beckmann et al., Solid St. Nuc. Mag. Res., 1998, 12, 251).

b. Preparation of 3-iso-propylchrysene.

(Z)-1-(4-isopropylstyryl)naphthalene (4 g, 7.34 mmol) was dissolved in 1 L of dry carbon tetrachloride in a 1 L photochemical vessel, equipped with an air inlet and a stirbar. Two condensers were attached on top of the photochemical vessel. Reaction mixture was irradiated with the halogen lamp (Hanovia, 450 W) for 4 hours. Volatiles were removed under vacuum and the resulting solids were extracted with toluene to give 1.6 g (40%) of a yellow powder. ¹H NMR (CD₂Cl₂, LIMS 643357): □ 1.32 (d, 6H, J=6.8 Hz), 3.02 (sept, 1H, J=6.8 Hz), 7.34-7.47 (m, 3H), 7.73-7.79 (m, 4H), 8.44 (d, 1H, J=9.3 Hz), 8.52 (s, 1H), 8.53 (d, 1H, J=9.2 Hz), 8.6 (d, 1H, J=9.2 Hz).

c. Preparation of 6,12-dibromo-3-iso-propylchrysene

Bromination was carried out as described in Kodomari et al., J. Org. Chem., 1988, 2093. Yield 22.5%. ¹H NMR (CD₂Cl₂): δ 1.21 (d, 6H, J=6.8 Hz), 2.83 (sept, 1H, J=6.8 Hz), 7.28-7.39 (m, 3H), 8.08 (d, 1H, J=8.4 Hz), 8.22 (s, 1H), 8.37 (d, 1H, J=8.8 Hz), 8.39 (dd, 1H, J=1.2, 8.2 Hz), 8.73 (s,1H), 8.94 (s,1H).

d. Preparation of E1

In a drybox, 6,12-dibromo-3-iso-propylchrysene (0.55 g, 1.28 mmol) and bis(3,4-dimethylphenyl)amine (0.593 g, 2.63 mmol) were combined in a thick-walled glass tube and dissolved in 15 ml of toluene. Tris(tert-butyl)phosphine (0.052 g, 0.26 mmol) and tris(dibenzylideneacetone)dipalladium(0) (0.118 g, 0.13 mmol) were added next, followed by sodium tert-butoxide (0.296 g, 3.08 mmol) and 5 ml of dry toluene. Glass tube was sealed, brought out of the box and placed into a 100° C. oil bath for 24 hours. Reaction mixture was cooled to room temperature, diluted with 80 ml of dichloromethane and filtered through a plug of celite and silica. The plug was washed with additional 200 ml of dichloromethane. Filtrates were combined and volatiles were removed under reduced pressure to give dark yellow solid. Further purification was done by column chromatography on silica using 10% dichloromethane in hexane. Second eluted fraction gave 750 mg (81%) of a bright yellow powder after evaporation. ¹H NMR (CD₂Cl₂): δ 1.22 (d, 6H, J=7 Hz), 2.03 (S, 12H), 2.10 (s, 12H), 3.02 (sept, 1H, J=7 Hz), 6.69-6.77 (m, 4H), 6.82 (dd, 4H, J=2.1, 6.5 Hz), 6.87 (dd, 4H, J=2.7, 8.2 Hz), 7.98 (d, 1H, J=8.4 Hz), 8.03 (d, 1H, J=8.4 Hz), 8.27 (s, 1H), 8.36 (s, 1H), 8.43-8.47 (m, 2H).

### Example 2

This example illustrates the preparation of Compound E2, 3-tert-butyl-N⁶,N⁶,N¹²,N¹²-tetrakis(3,4-dimethylphenyl)-chrysene-6,12-diamine

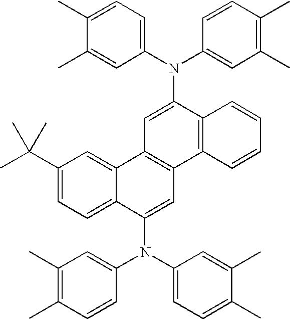

a. Preparation of 1-(4-tert-butylstyryl)naphthalenes.

An oven-dried 500 ml three-neck round bottom flask was equipped with a magnetic stir bar, addition funnel and nitrogen inlet connector. The flask was charged with (1-napthylmethyl)triphenylphosphonium chloride (12.07 g, 27.5 mmol) and 200 ml of anhydrous THF. Sodium hydride (1.1 g, 25 mmol) was added in one portion. The mixture became bright orange and was left to stir overnight at room temperature. A solution of 4-tert-butyl-benzaldehyde (7.1 g, 25 mmol) in anhydrous THF (30 ml) was added to the addition funnel with a cannula. The aldehyde solution was added to the reaction mixture dropwise over 45 minutes. Reaction was left to stir at room temperature for 24 hours (orange color went away). Silica gel was added to the reaction mixture and volatiles were removed under reduced pressure. The crude product was purified by column chromatography on silica gel using 5-10% dichloromethane in hexanes. The product was isolated as a mixture of cis- and trans-isomers (6.3 g, 89%) and used without separation. ¹H NMR (CD₂Cl₂): δ 1.27 (s, 9H), 7.08 (d, 1H, J=16 Hz), 7.33-7.49 (m, 7H), 7.68 (d, 1H, J=7.3 Hz), 7.71 (d, 1H, J=8.4 Hz), 7.76-7.81 (m, 2H), 8.16 (d,1H, J=8.3 Hz).

b. Preparation of 3-tert-butylchrysene.

1-(4-tert-Butylstyryl)naphthalenes (4.0 g, 14.0 mmol) were dissolved in dry toluene (1 l) in a one-liter photochemical vessel, equipped with nitrogen inlet and a stirbar. A bottle of dry propylene oxide was cooled in ice-water before 100 ml of the epoxide was withdrawn with a syringe and added to the reaction mixture. Iodine (3.61 g, 14.2 mmol) was added last. Condenser was attached on top of the photochemical vessel and halogen lamp (Hanovia, 450 W) was turned on. Reaction was stopped by turning off the lamp when no more iodine was left in the reaction mixture, as evidenced by the disappearance of its color. The reaction was complete in 3.5 hours. Toluene and excess propylene oxide were removed under reduced pressure to yield a dark yellow solid. Crude product was dissolved in a small amount of 25% dichloromethane in hexane, passed through a 4″ plug of neutral alumina, and washed with 25% dichloromethane in hexane (about one liter). Volatiles were removed to give 3.6 g (91%) of 3-tert-butylchrysene as a yellow solid. ¹H NMR (CD₂Cl₂): δ 1.41 (s, 9H), 7.51 (app t, 1H), 7.58 (app t, 1H), 7.63 (dd(1H, J=1.8, 8.4 Hz), 7.80-7.92 (m, 4H), 8.54 (d, 1H, J=9.1 Hz), 8.63-8.68 (m, 3H).

c. Preparation of 6,12-dibromo-3-tert-butylchrysene

3-tert-Butylchrysene (4.0 g, 14.1 mmol) and trimethylphosphate (110 ml) were mixed in a 500 ml round-bottom flask. After bromine (4.95 g, 31 mmol) was added, a reflux condenser was attached to the flask and reaction mixture was stirred for one hour in an oil bath at 105° C. A white precipitate formed almost immediately. Reaction mixture was worked up by pouring it onto a small amount of ice water (100 ml). The mixture was vacuum-filtered and washed well with water. The resulting tan solid was dried under vacuum. The crude product was boiled in methanol (100 ml), cooled to room temperature and filtered again to yield 3.74 g (60%) of a white solid. ¹H NMR (CD₂Cl₂): δ 1.46 (s, 9H), 7.70 (m, 2H), 7.79 (dd,1H, J=1.9, 8.8 Hz), 8.28 (d, 1H, J=8.7 Hz), 8.36 (dd, 1H, J=1.5, 8.0), 8.60 (d, 1H, J=1.8 Hz), 8.64 (dd, 1H, J=1.5, 8.0 Hz), 8.89 (s,1H), 8.97 (s,1H).

d. Preparation of 3-tert-butyl-N⁶,N^{1 2}-bis(3,4-dimethylphenyl)chrysene-6,12-diamine.

In a drybox, 6,12-dibromo-3-tert-butylchrysene (9.7 g, 21.94 mmol) and 3,4-dimethylaniline (5.58 g, 46.1 mmol) were combined in a 500 ml round-bottom flask and dissolved in 200 ml of dry toluene. Tris(tert-butyl)phosphine (0.080 g, 0.395 mmol) and tris(dibenzylideneacetone)dipalladium(0) (0.180 g, 0.197 mmol) were dissolved in 25 ml of dry toluene and stirred for 10 minutes. The catalyst solution was added to the reaction mixture, stirred for 10 minutes and followed by sodium tert-butoxide (4.2 g, 43.9 mmol) and 100 ml of dry toluene. After another 10 minutes, the reaction flask was brought out of the drybox, attached to a nitrogen line and stirred at 80° C. overnight. Next day, reaction mixture was cooled to room temperature and filtered through a 4 inch plug of silica gel and celite, washing with 1 liter of dichloromethane. Removal of volatiles under reduced pressure gave a dark brown solid. The crude product was further purified on a 500 g silica gel column (5″ wide, 7″ high) using a gradient of dichloromethane in hexanes (20% to 50%). Combined fractions yielded 10.5 g (91%) of product as a yellow solid. ¹H NMR (CD₂Cl₂): δ 1.39 (s, 9H), 2.15 (s, 6H), 2.16 (s, 6H), 5.94 (s, 1H), 5.98 (s, 1H), 6.70 (dd, 1H), 6.79 (dd, 1H), 6.86 (app dd, 2H), 6.97 (app dd, 2H), 7.48-7.58 (m, 2H), 7.63 (dd, 1H), 8.01 (d, 1H), 8.05 (dd, 1H), 8.34 (s,1H), 8.46-8.53 (m, 3H).

e. Preparation of E2

In a drybox, 3-tert-butyl-N⁶,N¹²-bis(3,4-dimethylphenyl)chrysene-6,12-diamine (5.0 g, 9.6 mmol) and 4-bromo-o-xylene (4.1 g, 22.2 mmol) were combined in a 500 ml round-bottom flask and dissolved in 125 ml of dry toluene. Tris(tert-butyl)phosphine (0.037 g, 0.182 mmol) and tris(dibenzylideneacetone) dipalladium(0) (0.083 g, 0.091 mmol) were dissolved in 50 ml of dry toluene and stirred for 10 minutes. The catalyst solution was added to the reaction mixture, stirred for 10 minutes and followed by sodium tert-butoxide (2.1 g, 22.2 mmol) and 50 ml of dry toluene. After another 10 minutes, the reaction flask was brought out of the drybox, attached to a nitrogen line and stirred at 80° C. overnight. Next day, reaction mixture was cooled to room temperature and filtered through a 4 inch plug of silica gel and celite, washing with 0.5 liter of dichloromethane. Removal of volatiles under reduced pressure gave 7.25 g of a yellow solid. A portion of the crude product (3.5 g) was purified further on a 110 g Florosil® column using a gradient of dichloromethane in hexanes (10% to 40%). Removal of volatiles yielded 2.5 g (72.4%) of product as a pale yellow solid. ¹H NMR (CD₂Cl₂): δ 1.31 (s, 9H), 2.037 (s, 3H), 2.044 (s, 3H), 2.11 (br s, 6H), 6.73 (app t, 4H), 6.82 (app d, 4H), 6.89 (app dd, 4H), 7.39 (app t, 1H), 7.45-7.52 (m, 2H), 7.97 (d, 1H), 8.03 (d, 1H), 8.35-8.48 (m, 4H).

### Example 3

This example illustrates the preparation of Compound E3, 3,3′-(3-tert-butylchrysene-6,12-diyl)bis((3,4-dimethylphenyl)azanediyl)-dibenzonitrile.

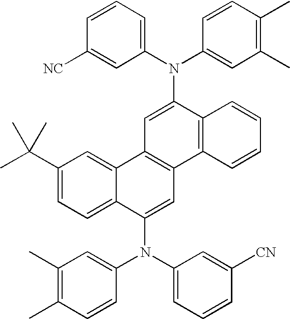

In a drybox, 3-tert-butyl-N⁶,N¹²-bis(3,4-dimethylphenyl)chrysene-6,12-diamine (2.03 g, 3.88 mmol) and 3-bromobenzonitrile (1.45 g, 7.95 mmol) were placed into a thick-walled glass tube and dissolved in 25 ml of dry toluene. Tris(tert-butyl)phosphine (0.031 g, 0.16 mmol) and tris(dibenzylideneacetone) dipalladium(0) (0.071 g, 0.08 mmol) were added next, followed by sodium tert-butoxide (0.894 g, 9.31 mmol) and 5 ml of dry toluene. Glass tube was sealed, brought out of the drybox and placed into a 90° C. oil bath for 16 hours. Next day, reaction mixture was cooled to room temperature. Diluted with dichloromethane (150 ml) and filtered through a 4 inch plug of silica gel and celite, washing with 0.5 liter of dichloromethane. Removal of volatiles under reduced pressure gave dark red solid. The crude product was further purified on a silica gel column (2″ wide, 6″ high) using a 1:1 v/v dichloromethane in hexanes. Three fractions were collected and the middle one was further purified by filtering through a 1×2″ inches Florosil® plug using dichloromethane. The resulting yellow solution was concentrated under reduced pressure to give 0.97 g (34.5%) of a yellow solid (LC showed no improvement in purity over the sample before florosil column). ¹H NMR (CD₂Cl₂): δ 1.34 (s, 9H), 2.09 (s, 3H), 2.10 (s, 3H), 2.15 (br s, 6H), 6.9-7.18 (m, 14 H), 7.45 (app t, 1H), 7.56 (m, 2H), 7.93 (d, 1H), 8.45 (br d, 1H), 8.46 (br s, 1H), 8.52 (m, 2H).

### Example 4

This example illustrates the preparation of Compound E4, 3-tert-butyl-N⁶,N¹²-bis(4-tert-butylphenyl)-N⁶,N¹²-bis(3,4-dimethylphenyl) chrysene-6,12-diamine.

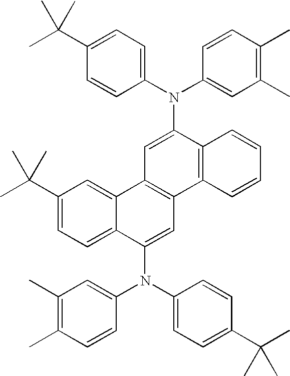

In a drybox, 3-tert-butyl-N⁶,N¹²-bis(3,4-dimethylphenyl)chrysene-6,12-diamine (3.3 g, 6.3 mmol) and 1-bromo-4-tert-butylbenzene (2.96 g, 13.9 mmol) were combined in a 500 ml round-bottom flask and dissolved in 125 ml of dry toluene. Tris(tert-butyl)phosphine (0.023 g, 0.11 mmol) and tris(dibenzylideneacetone) dipalladium(0) (0.052 g, 0.057 mmol) were dissolved in 5 ml of dry toluene and stirred for 10 minutes. The catalyst solution was added to the reaction mixture, stirred for 10 minutes and followed by sodium tert-butoxide (1.33 g, 13.91 mmol) and 40 ml of dry toluene. After another 10 minutes, the reaction flask was brought out of the drybox, attached to a nitrogen line and stirred at 80° C. overnight. Next day, reaction mixture was cooled to room temperature and filtered through a 4 inch plug of silica gel and one inch of celite, washing with 1.2 liters of chloroform. Removal of volatiles under reduced pressure gave 5.6 g of a yellow solid. A portion of the crude product (3.5 g) was purified further on a 200 g silica gel column using a gradient of chloroform in hexanes (5% to 20%). Removal of volatiles yielded 4.5 g (90.5%) of product as a pale yellow solid. ¹H NMR (CD₂Cl₂): δ 1.20 (s, 9H), 1.21 (s, 9H), 1.30 (s, 9H), 2.05 (br s, 3H), 2.06 (br s, 3H), 2.16 (br s, 6H), 6.78 (app t, 2H), 6.91 (m, 8 H), 7.13 (app d, 2H), 7.16 (app d, 2H), 7.40 (app t, 1H), 7.50 (m, 2H), 7.97 (d, 1H), 8.04 (d, 1H), 8.38 (d, 1H), 8.42 (br d, 2H), 8.48 (d, 1H).

### Example 5

This example illustrates the preparation of Compound E5, N⁶, N¹²-bis(biphenyl-4-yl)-3-tert-butyl-N⁶,N¹²-bis(4-tert-butylphenyl)chrysene-6,12-diamine.

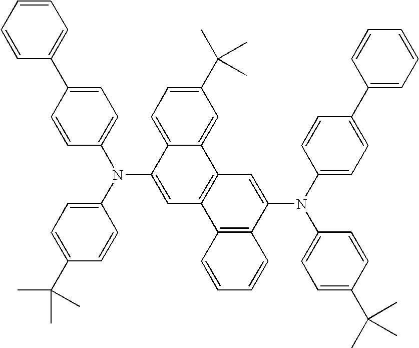

In a drybox, 3-tert-butyl-6,12-dibromochrysene (1.8 g, 4.07 mmol) and N-(4-tert-butylphenyl)biphenyl-4-amine (2.58 g, 8.55 mmol) were combined in a thick-walled glass tube and dissolved in 20 ml of dry toluene. Tris(tert-butyl)phosphine (0.0148 g, 0.073 mmol) and tris(dibenzylideneacetone) dipalladium(0) (0.0335 g, 0.0366 mmol) were dissolved in 10 ml of dry toluene and stirred for 10 minutes. The catalyst solution was added to the reaction mixture, stirred for 10 minutes and followed by sodium tert-butoxide (0.782 g, 8.14 mmol) and 20 ml of dry toluene. After another 10 minutes, the reaction flask was brought out of the drybox, attached to a nitrogen line and stirred at 80° C. overnight. Next day, reaction mixture was cooled to room temperature and filtered through a 4 inch plug of silica gel and one inch of celite, washing with one liter of chloroform and 300 ml of dichloromethane. Removal of volatiles under reduced pressure gave a yellow solid. The crude product was purified further by silica gel column chromatography using a gradient of dichloromethane in hexanes (10% to 15%). Removal of volatiles yielded 3.25 g (90.5%) of product as a yellow solid. ¹H NMR (CD₂Cl₂): δ 1.22 (s, 9H), 1.23 (s, 9H), 1.31 (s, 9H), 7.04-7.56 (m, 29 H), 8.00 (d, 1H, J=8.8 Hz), 8.07 (dd, 1H, J=1.1, 8.3 Hz), 8.44 (d, 1H, J=1.8 Hz), 8.51 (s, 1H), 8.53 (s,1H), 8.54 (d, 1H, J=8.3 Hz).

### Example 6

This example illustrates the synthesis of Compound E6, 3-tert-Butyl-6,12-N,N′-bis(4-tert-butylphenyl)-6,12-N,N′-bis(m-fluorophenyl)chrysenediamine.

Compound E6 was prepared from 3-tert-butyl-6,12-dibromochrysene and N-(4-tert-butylphenyl)-3-fluoroaniline as described in Example 5. Yield 820 mg (84%). ¹H NMR (dmf-d₇): □ 1.32 (s, 9H), 1.33 (S, 9H), 1.45 (s, 9H), 6.69-6.76 (m, 4H), 6.78-6.83 (m, 2H), 7.28-7.40 (m, 6H), 7.45-7.50 (m, 4H), 7.68 (ddd, 1H, J=1.0, 6.9, 9.3 Hz), 7.76 (ddd, 1H, J=1.4, 6.9, 8.4 Hz), 7.82 (dd, 1H, J=1.8, 8.7 Hz), 8.18 (d, 1H, J=8.7Hz), 8.20 (dd, 1H, J=1.0, 8.4 Hz), 8.86 (d, 1H, J=1.7 Hz), 8.97 (s, 1H), 9.0 (d,1H, J=8.4 Hz), 9.05 (s,1H).

### Example 7

This example illustrates the synthesis of Compound E7, 3-tert-Butyl-6,12-N,N′-bis(4-tert-butylphenyl)-6,12-N,N′-bis(o-tolyl)chrysenediamine.

Compound E7 was prepared from 3-tert-butyl-6,12-dibromochrysene and N-(o-tolyl)-4-tert-butylaniline as described in Example 6. Crude product was purified by trituration with hexane and diethyl ether. Yield 700 mg (78%). ¹H NMR (dmf-d₇): □ 1.26 (s, 9H), 1.28 (s, 9H), 1.32 (s, 9H), 2.13 (s, 3H), 2.17 (s, 3H), 6.74 (d, 2H, J=8.4 Hz), 6.79 (d, 2H, J=8.6 Hz), 7.14-7.39 (m, 12 H), 7.50 (app t, 1H, J=7.8 Hz), 7.61 (app t, 1H, J=7.6 Hz), 7.65 (dd, 1H, J=1.7, 8.4 Hz), 8.04 (d, 1H, J=8.9 Hz), 8.07 (dd,1H, J=0.8, 8.4 Hz), 8.31-8.34 (m, 2H), 8.35 (s, 1H), 8.49 (d, 1H, J=8.4 Hz).

### Example 8

This example illustrates the synthesis of Compound E8, 3-tert-Butyl-6,12-N,N′-bis(4-biphenyl)-6,12-N,N′-bis(m-fluorophenyl)chrysenediamine.

Compound E8 was prepared from 3-tert-butyl-6,12-dibromochrysene and N-(m-fluorophenyl)-4-biphenylamine as described in Example 6. Yield 930 mg (79.6%). ¹H NMR (dmf-d₇): □ 1.41 (s, 9H), 6.79 (app dt, 2H, J=2.4, 8.4 Hz), 6.90 (br d, 2H, J=11.6 Hz), 6.96 (m, 2H), 7.28-7.40 (m, 8H), 7.45 (t, 4H, J=7.5 Hz), 7.62-7.75 (m, 10H), 7.80 (dd, 1H, J=1.8, 8.4 7Hz), 8.15 (d, 1H, J=8.8 Hz), 8.18 (dd, 1H, J=1.1, 8.3 Hz), 8.90 (d, 1H, J=1.6 Hz), 8.99 (s, 1H), 9.0 (d, 1H, J=8.3 Hz), 9.15 (s, 1H).

### Example 9

This example illustrates the synthesis of Compound E9, 3-tert-Butyl-6,12-N,N′-bis(4-tert-butylphenyl)-6,12-N,N′-bis(4-(1-naphthyl)phenyl)chrysenediamine.

Compound E9 was prepared from 3-tert-butyl-6,12-dibromochrysene and N-(4-(1-naphthyl)phenyl)-4-tert-butylaniline as described in Example 6. Crude product was purified by column chromatography with 5-12% CH₂Cl₂ in hexane. Yield 440 mg (33.6%). ¹H NMR (dmf-d₇): □ 1.29 (s, 9H), 1.30 (s, 9H), 1.43 (s, 9H), 7.23 (m, 4H), 7.31 (m, 4H), 7.41-7.46 (m,10H), 7.46-7.59 (m, 6H),7.66 (app t,1H, J=7.6 Hz), 7.75 (app t, ¹H, J=7.6 Hz), 7.81 (dd, 1H, J=1.8, 8.5 Hz), 7.93 (dd, 2H, J=3.3, 8.4 Hz), 8.25 (d, 1H, J=8.8 Hz), 8.27 (dd, 1H, J=1.1, 8.9 Hz), 8.83 (d, 1H, J=1.7 Hz), 8.98 (s, 1H), 8.99 (d, 1H, J=8.3 Hz), 9.03 (s, 1H).

TABLE 1
Solution Photoluminescence Data.

| | | | |
|---|---|---|---|
| | Solution PL | | |
| Example | Toluene, nm | CIE x | CIE y |

| | | | |
|---|---|---|---|
| E1 | 456 | 0.137 | 0.114 |
| E2 | 458 | 0.134 | 0.116 |
| E3 | 442 | 0.147 | 0.064 |
| E4 | 455 | 0.138 | 0.107 |
| E5 | 454 | 0.138 | 0.109 |
| E6 | 443 | 0.146 | 0.065 |
| E7 | 447 | 0.143 | 0.078 |
| E8 | 444 | 0.146 | 0.07 |
| E9 | 454 | 0.139 | 0.10 |

PL is photoluminescence

CIE x and y are the color coordinates according to the C.I.E. 20 chromaticity scale (Commision Internationale de L'Eclairage, 1931).

### Examples 10-21

These examples demonstrate the fabrication and performance of a device having blue emission. The following materials were used:

Indium Tin Oxide (ITO): 50 nm

buffer layer=Buffer 1 (25 nm), which is an aqueous dispersion of polypyrrole and a polymeric fluorinated sulfonic acid. The material was prepared using a procedure similar to that described in Example 1 of published U.S. patent application no. 2005/0205860.

hole transport layer=polymer P1 (20 nm)

photoactive layer=13:1 host:dopant (48 nm)

electron transport layer=Tetrakis-(8-hydroxyquinoline) zirconium (ZrQ) (20 nm)

cathode=LiF/Al (0.5/100 nm)

OLED devices were fabricated by a combination of solution processing and thermal evaporation techniques. Patterned indium tin oxide (ITO) coated glass substrates from Thin Film Devices, Inc were used. These ITO substrates are based on Corning 1737 glass coated with ITO having a sheet resistance of 30 ohms/square and 80% light transmission. The patterned ITO substrates were cleaned ultrasonically in aqueous detergent solution and rinsed with distilled water. The patterned ITO was subsequently cleaned ultrasonically in acetone, rinsed with isopropanol, and dried in a stream of nitrogen.

Immediately before device fabrication the cleaned, patterned ITO substrates were treated with UV ozone for 10 minutes. Immediately after cooling, an aqueous dispersion of Buffer 1 was spin-coated over the ITO surface and heated to remove solvent. After cooling, the substrates were then spin-coated with a solution of a hole transport material, and then heated to remove solvent. After cooling the substrates were spin-coated with the emissive layer solution, and heated to remove solvent. The substrates were masked and placed in a vacuum chamber. A ZrQ layer was deposited by thermal evaporation, followed by a layer of LiF. Masks were then changed in vacuo and a layer of Al was deposited by thermal evaporation. The chamber was vented, and the devices were encapsulated using a glass lid, dessicant, and UV curable epoxy.

The OLED samples were characterized by measuring their (1) current-voltage (I-V) curves, (2) electroluminescence radiance versus voltage, and (3) electroluminescence spectra versus voltage. All three measurements were performed at the same time and controlled by a computer. The current efficiency of the device at a certain voltage is determined by dividing the electroluminescence radiance of the LED by the current density needed to run the device. The unit is a cd/A. The power efficiency is the current efficiency divided by the operating voltage. The unit is Im/W. The device data is given in Table 2.

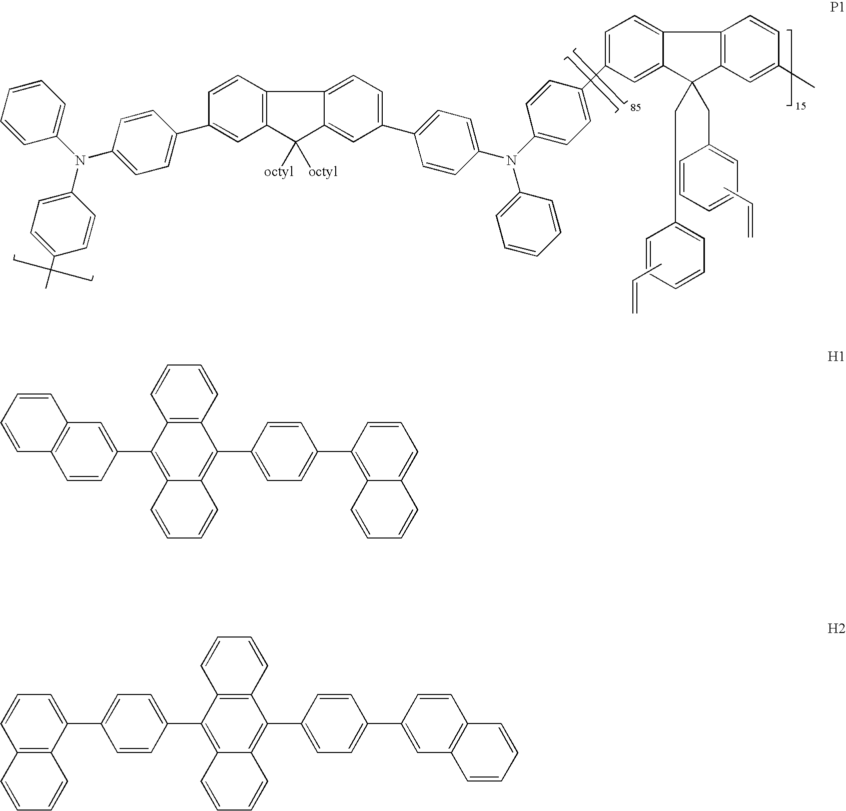

### Example 10

In this example, the blue dopant was Compound E1, and the host was H1.

### Example 11

In this example, the blue dopant was E2, and the host was H1.

### Example 12

In this example, the blue dopant was E3, and the host was H1.

### Example 13

In this example, the blue dopant was E2, and the host was H2.

### Example 14

In this example, the blue dopant was Compound E4, and the host was H1.

### Example 15

In this example, the blue dopant was Compound E4, and the host was H2.

### Example 16

In this example, the blue dopant was Compound E5, and the host was H1.

### Example 17

In this example, the blue dopant was Compound E5, and the host was H2.

### Example 18

In this example, the blue dopant was Compound E6, and the host was H1.

### Example 19

In this example, the blue dopant was Compound E7, and the host was H1.

### Example 20

In this example, the blue dopant was Compound E8, and the host was H1.

### Example 21

In this example, the blue dopant was Compound E9, and the host was H1.

TABLE 2

| | | | | | Lum. |
|---|---|---|---|---|---|
| Example | CE [cd/A] | Voltage (V) | CIE [x] | CIE [y] | ½ Life [h] |
| 10 | 6.0 | 7.0 | 0.14 | 0.20 | 1090 |
| 11 | 5.9 | 5.6 | 0.136 | 0.159 | 1110 |
| 12 | 1.7 | 5.7 | 0.149 | 0.133 | 83 |
| 13 | 5.8 | 5.9 | 0.137 | 0.160 | 1920 |
| 14 | 5.4 | 5.9 | 0.137 | 0.164 | 1300 |
| 15 | 5.8 | 5.8 | 0.137 | 0.161 | 1920 |
| 16 | 4.3 | 5.4 | 0.137 | 0.152 | 3020 |
| 17 | 4.4 | 5.7 | 0.137 | 0.154 | 3020 |
| 18 | 1.5 | 6.3 | 0.146 | 0.116 | 200 |
| 19 | 3.4 | 5.6 | 0.142 | 0.134 | 800 |
| 20 | 1.4 | 6.4 | 0.147 | 0.116 | 210 |
| 21 | 3.4 | 5.6 | 0.140 | 0.137 | 1650 |

* All data @ 2000 nits, CE = current efficiency, CIE x and y are the color coordinates according to the C.I.E. chromaticity scale (Commision Internationale de L'Eclairage, 1931).

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

The use of numerical values in the various ranges specified herein is stated as approximations as though the minimum and maximum values within the stated ranges were both being preceded by the word “about.” In this manner slight variations above and below the stated ranges can be used to achieve substantially the same results as values within the ranges. Also, the disclosure of these ranges is intended as a continuous range including every value between the minimum and maximum average values including fractional values that can result when some of components of one value are mixed with those of different value. Moreover, when broader and narrower ranges are disclosed, it is within the contemplation of this invention to match a minimum value from one range with a maximum value from another range and vice versa.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination. Further, reference to values stated in ranges includes slight variations above and below the stated ranges can be used to achieve substantially the same results as values within the ranges. Also, the disclosure of these ranges is intended as a continuous range including every value between the minimum and maximum average values including fractional values that can result when some of components of one value are mixed with those of different value. Moreover, when broader and narrower ranges are disclosed, it is within the contemplation of this invention to match a minimum value from one range with a maximum value from another range and vice versa.

What is claimed is:

1. A compound having Formula I: 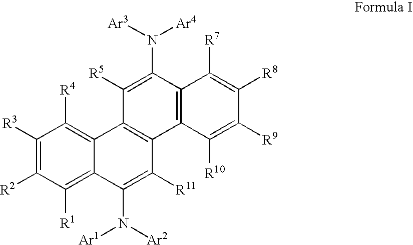
wherein:
Ar1 through Ar4 are the same or different and are aryl, and at least one of Ar1 through Ar4 is substituted;
R1 through R5 and R7 through R11 are the same or different and are selected from the group consisting of H and a branched alkyl, or adjacent R groups may be joined together to form a 5- or 6-membered aliphatic ring, with the proviso that either (i) R3 is a branched alkyl or (ii) R2 and R3 together form a 5- or 6-membered aliphatic ring;
wherein said compound is capable of emitting blue light.

2. The compound of claim 1, wherein the branched alkyl group has from 3-8 carbon atoms.

3. The compound of claim 1, wherein the branched alkyl group is selected from the group consisting of isopropyl, 2-butyl, t-butyl and 2-(2-methyl)-butyl.

4. The compound of claim 1, wherein R3 is a branched alkyl group.

5. The compound of claim 4, wherein R1, R2, R5, and R7 through R11 are H.

6. The compound of claim 1, wherein R2 and R3 taken together form an aliphatic ring selected from the group consisting of cyclopentyl and cyclohexyl.

7. The compound of claim 1, wherein Ar1 through Ar4 are independently selected from the group consisting of phenyl, biphenyl, naphthyl, and binaphthyl.

8. The compound of claim 7, wherein at least one of Ar1 through Ar4 has at least one substituent selected from alkyl, alkoxy, perfluoroalkyl, cyano, and fluoro.

9. The compound of claim 1, wherein Ar1 and Ar3 are phenyl.

10. The compound of claim 9, wherein Ar1 and Ar3 have one substituent selected from the group consisting of perfluoroalkyl, cyano, and fluoro.

11. The compound of claim 9, wherein Ar1 and Ar3 have 1-5 substituents selected from the group consisting of alkyl groups and alkoxy groups.

12. The compound of claim 1, wherein Ar2 and Ar4 are selected from the group consisting of phenyl groups and biphenyl groups.

13. The compound of claim 12, wherein Ar2 and Ar4 have at least one alkyl substituent.

14. An organic electronic device comprising a first electrical contact layer, a second electrical contact layer, and at least one active layer therebetween, wherein the active layer comprises a compound having Formula I: 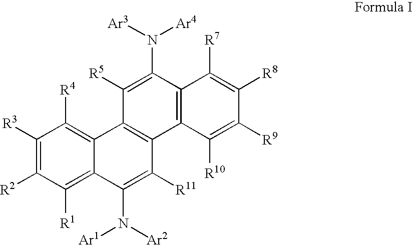
wherein:
Ar1 through Ar4 are the same or different and are aryl, and at least one of Ar1 through Ar4 is substituted;
R1 through R5 and R7 through R11 are the same or different and are selected from the group consisting of H and a branched alkyl, or adjacent R groups may be joined together to form a 5- or 6-membered aliphatic ring, with the proviso that either (i) R3 is a branched alkyl or (ii) R2 and R3 together form a 5- or 6-membered aliphatic ring;
wherein said compound is capable of emitting blue light.

15. An active layer comprising a compound of claim 1.

16. The active layer of claim 15 further comprising a host material.

17. The active layer of claim 15 wherein the host material has the formula,
An-L-An
where:
An is an anthracene moiety;
L is a divalent connecting group.

18. The active layer of claim 15 wherein the host material has the formula,
A-An-A
where:
An is an anthracene moiety;
A is the same or different at each occurrence and is an aromatic group.

19. The active layer of claim 18 wherein the host has the formula: 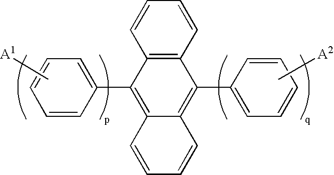
where:
A¹ and A² are the same or different at each occurrence and are selected from the group consisting of H, an aromatic group, and an alkenyl group, or A may represent one or more fused aromatic rings;
p and q are the same or different and are an integer from 1-3.

20. The active layer of claim 16 wherein the host is selected from the group consisting of 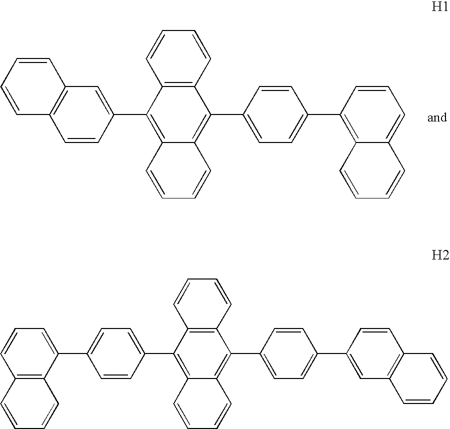
and combinations thereof.
